(19) United States
(12) Patent Application Publication
(10) Pub. No.: US 2014/0185010 A1
(43) Pub. Date: Jul. 3, 2014

(54) METHOD AND A DEVICE FOR MONITORING PUPIL

(71) Applicant: IDMED, Marseille (FR)

(72) Inventors: Frederic BERNERT, Marseille (FR); Thierry BAGNOL, Marseille (FR)

(73) Assignee: IDMED, Marseille (FR)

(21) Appl. No.: 13/727,924

(22) Filed: Dec. 27, 2012

(51) Int. Cl. A61B 3/11; A61B 3/00

(52) U.S. Cl. CPC A61B 3/112; A61B 3/00
USPC 351/219; 351/246

(57) ABSTRACT

The invention provides a method of monitoring the pupil of a subject, wherein a sensor for observing the pupil is arranged between the cornea and the eyelid covering the cornea, the sensor is powered through the eyelid, and the pupil-observation signals delivered by the sensor are collected through the eyelid.

### TECHNICAL FIELD

The present invention relates to a method and to a device for monitoring the pupil of a human subject.

### STATE OF THE ART

It is known that the pupil of a human subject is an indicator of the physiological or psychological state of the subject.

It is observed in particular that the size of the pupil varies as a function of numerous parameters in the subject's environment and external stimulations, and also as a function of the state of the subject, such as ambient light level, sensitivity to pain, taking drugs or analgesics, wakefulness, or an emotional state. Pupil dilatation and contraction reflexes also depend on the state of the subject, and in particular on diseases of the subject.

Measuring the size of the pupil is thus advantageous in various fields of medicine such as addictology, anesthesia, ophthalmology, neurology, psychology, pharmacology, intensive care, or toxicology.

Measurements of the pupil may be performed using a pupillometer programmed to measure variations in the size or the shape of the pupil, without putting any object into contact with the eyeball, as described for example in patent application US 2009/0174865.

In order to take such a measurement, the subject's eyelids are kept open and images of the subject's eye are obtained, which eye may be stimulated by light, emotion, or pain, and then the size (e.g. the area or the diameter) of the pupil is determined as a function of the images.

That type of appliance can be used for periods of short duration only (a few minutes). It is not possible to keep an eye open for a longer period without running the risk of drying the eye.

U.S. Pat. No. 5,297,554 and U.S. Pat. No. 5,903,333 describe appliances for measuring the size of the pupil, which appliances comprise a lens in contact with the eye and secured to a tubular extension, enabling the eye to be filmed using a separate optical appliance.

U.S. Pat. No. 4,007,980 describes an appliance that comprises a transparent body adhering to the eye in order to follow the movements of the eye. The body has light-emitting diodes (LEDs) fastened thereto for stimulation purposes that emit visible light, LEDs for illuminating the iris that emit invisible light (infrared light), and photodiodes that are sensitive to the invisible light in order to deliver a signal as a function of which the size of the pupil is determined.

The use of such appliances likewise presents the drawback of the eye being kept open, which goes against medical practices for monitoring subjects who are unconscious. In addition, it may be necessary to deliver artificial tears, thereby making monitoring more complicated to perform.

### SUMMARY OF THE INVENTION

An object of the invention is to propose a sensor and a device including the sensor in particular for monitoring variations—over time—in the size of the pupil of a human subject, in a manner that is simple, comfortable, and without danger for the subject, for a duration that may be long (of the order of at least one or several hours, e.g. of the order of one or several days).

An object of the invention is to propose a sensor for monitoring the pupil of a subject, a device including the sensor, and a monitoring method, for improving and/or remedying, at least in part, the shortcomings or the drawbacks of known pupil-monitoring systems.

According to an aspect of the invention, there is provided a sensor arranged to rest on the cornea and/or the sclera of an eye of a subject, and under the eyelid(s) covering the eye, and to deliver signals through the eyelid(s), which signals are characteristic in particular of the size of the pupil of the eye.

In another aspect of the invention, there is provided a device for monitoring the pupil of an eye of a subject, the device comprising a sensor adapted to be inserted between the eyeball and the upper eyelid—or the eyelids—of the eye, a signal processor unit for processing the signals delivered by the sensor that is arranged in particular to determine the size of the pupil of the eye as a function of said signals, and signal transport means for transporting signals through the eyelid(s) and connecting the sensor to the signal processor unit while allowing the subject's eyelids to be kept closed.

In another aspect of the invention, there is provided a method of monitoring the pupil of a subject, wherein a pupil-observation sensor is arranged between the cornea and the eyelid(s) covering the cornea, the sensor is powered through the eyelid(s), and the pupil-observation signals delivered by the sensor are collected through the eyelid(s).

For this purpose, the sensor comprises photoelectric detectors—or light receivers—, in particular detectors that are sensitive to infrared radiation, which sensors are arranged to “observe” the pupil, i.e. to be sensitive to light reflected by the portion of the iris that surrounds the pupil.

The photoelectric detectors—or light receivers—may be photodiodes, phototransistors, complementary metal oxide semiconductor (CMOS) sensors, or charge-coupled devices (CCDs). They may be grouped together in large numbers in order to form a matrix imager, or they may constitute components that are isolated (discrete).

The sensor includes a signal transmission device for transmitting signals from the light receivers through the eyelid(s), which device includes electrical conductors connected to the light receivers.

These electrical conductors may be arranged in a waterproof covering or they may be covered with a waterproof coating.

The electrical conductors serve to transmit the electrical energy needed for powering the light receivers, and also to transmit the signals delivered thereby.

The sensor comprises a body of flat shape containing the light receivers and at least some of the conductors, the thickness of the body being small, preferably less than or equal to about 4 millimeters (mm), and in particular lying in a range about 0.3 mm to about 4 mm.

The body of the sensor may be generally in the shape of a spherical cap having two main outer faces: a concave proximal face adapted to be in contact with the cornea and/or the sclera, and a convex distal face adapted to be in contact with the eyelid covering the sensor and/or the cornea.

The portions of the body that extend between the light receivers and the proximal face are generally transparent to infrared radiation so that when the iris is illuminated with such radiation, the radiation that is reflected by the iris is transmitted to the light receivers through these portions of the body, and the signals delivered by the light receivers in response to detecting the reflected IR radiation enable an image of the pupil to be formed and/or enable its diameter to be measured.

The body of the sensor may include two superposed walls: a distal wall adapted to support a portion of the eyelid(s), and a proximal wall that is transparent at least in part and that is adapted to rest against the cornea and/or the sclera.

The light receivers and the conductors may extend, at least in part, between these two walls or in cavities formed in at least one of these walls.

Each of these walls may be generally in the shape of a spherical cap.

The proximal wall that forms the base of the sensor and that serves as a support for the light receivers may be constituted essentially by a contact lens—or ophthalmic lens—that is generally non-correcting.

The contact lens may be made of an oxygen-permeable material, in particular a material containing a silicone-based polymer, such as a silicone hydrogel, in order to form a soft lens, or else in a material that is harder, e.g. containing methylmethacrylate, and in particular polymethylmethacrylate (PMMA).

In other words, and in another aspect of the invention, there is provided a light-monitoring sensor comprising a transparent base—such as a contact lens—presenting a concave first face adapted to rest on the cornea and/or the sclera and an opposite second face—in particular a convex second face that is substantially parallel to the concave first face—; the sensor also includes light receivers arranged facing the second face of the transparent base, a signal transmission device that is connected to the light receivers, that extends facing the transparent base and/or along (or extending) said base, and that is arranged to take the signals from the light receivers and transmit them through the (closed) eyelids covering the eye; the sensor also includes a covering coating and/or covering the light receivers and at least a portion of the signal transmission member, the outer (or distal) face of said covering being adapted, in particular being convex, for coming into contact with—and/or for supporting—the inside face(s) of the closed eyelid(s).

The covering that covers and/or contains the light receivers may be transparent to visible radiation and/or to infrared radiation; in particular it may be made like a contact lens out of a soft or hard oxygen-permeable material.

Alternatively, the covering may be opaque to visible and/or infrared radiation.

The transparent base and/or the covering may also include a waterproof film covering the light receivers.

An optical system—such as a lens—is arranged between the concave face of the transparent base and the light receivers so as to focus the light receivers substantially on the plane of the pupil (and of the iris).

The optical system may also include a diaphragm and/or a filter limiting the sensitivity of the light receivers to interfering radiation.

The optical system may be secured to the light receivers, or it may be integrated in the transparent base.

In an embodiment, the body of the sensor may be constituted essentially by a contact lens with a covering superposed thereon, with the light receivers and at least some of the members for transmitting signals through the eyelids being inserted between them.

The sensor may include sources of visible light and/or of infrared radiation, which sources are arranged facing the transparent base in order to illuminate the iris and/or produce visible stimuli, preferably at a distance from the light receivers: the light receivers are preferably arranged in a central portion of the sensor, and the light sources are arranged in a peripheral portion of the sensor.

In an embodiment, the sensor includes an extension extending the body of the sensor and containing electrical conductors that extend the conductors contained in the body. This extension may be in the from of a ribbon cable and it may present a thickness that is sufficiently small to be able to pass between the eyelids through the gap that exists between closed eyelids, substantially without deforming the eyelids or spacing them apart. This thickness may be of the order of about 50 micrometers (μm) to about 500 μm.

In another embodiment, the signal transmission member of the sensor comprises a wireless signal transmission module incorporated in the body of the sensor and connected to the electrical conductors in such a manner as to transmit power supply signals to the light receivers, which signals are delivered through the eyelid(s) covering the sensor by means of a power supply appliance separate from the sensor, and so as to transmit the signals delivered by the light receivers of the sensors to a signal processor unit that is separate from the sensor, said signals being transmitted through the eyelid(s) covering the sensor.

The wireless transmission of signals and/or power through the eyelid(s) may be performed in particular by radio waves, by infrared radiation, or by inductive coupling.

For this purpose, the sensor may include an antenna included in the body of the sensor and connected to the electrical conductors via the wireless signal transmission module incorporated in the sensor.

Other aspects, characteristics, and advantages of the invention appear from the following description which refers to the accompanying figures and illustrates preferred embodiments of the invention without any limiting character.

### BRIEF DESCRIPTION OF THE FIGURES

FIG. 1 is a diagram of the face of a subject and of a device for monitoring the pupil, with the sensor of the device resting under the upper eyelid of one of the subject's eyes.

FIG. 2 is a diagrammatic cross-section view of a pupil-monitoring sensor extending under the upper eyelid of a subject's eye, together with a portion of the monitoring device that includes the sensor.

FIG. 3 is a diagrammatic view similar to FIG. 2 showing a monitoring sensor extending under the upper eyelid and fitted with a wireless transmission member, together with a portion of the monitoring device that includes the sensor.

FIG. 4 is a diagrammatic cross-section view of the main components of a pupil-monitoring device, in another embodiment.

FIG. 5 is a diagrammatic cross-section view on a larger scale of the main components of the eye-monitoring sensor of FIG. 2.

FIG. 6 is a diagrammatic plan view showing the main components of the FIG. 4 eye-monitoring sensor.

FIG. 7 is a diagrammatic plan view of the main components of a pupil-monitoring sensor in another embodiment.

FIG. 8 is a diagrammatic plan view of the main components of a pupil-monitoring sensor in yet another embodiment.

FIG. 8A is a diagram similar to FIG. 5 showing a pupil-monitoring sensor in another embodiment.

FIG. 9 is a diagram similar to FIGS. 7 and 8 showing a pupil-monitoring sensor in another embodiment.

FIG. 10 is a diagram in cross-section view of another embodiment of an eye-monitoring device including a wireless transmission sensor, using an induction loop for transmitting energy and an IR emitter/receiver for transmitting signals.

FIGS. 11 to 13 show a variant embodiment in which the light receivers of the sensor are arranged in line: FIG. 11 is a diagrammatic plan view; FIG. 12 is a diagrammatic cross-section view; and FIG. 13 illustrates an operation of monitoring the diameter of the pupil on the basis of data delivered by the light receivers.

FIGS. 14 to 16 show a variant embodiment in which a miniature camera and LEDs of the sensor are arranged in line: FIG. 14 is a diagrammatic view of the sensor in cross-section; FIG. 16 is a diagrammatic plan view of the sensor; and FIG. 15 is a plan view showing a flexible printed circuit that is common to the camera and to the LEDs.

### DETAILED DESCRIPTION OF THE INVENTION

As described in greater detail below, a system is proposed for prolonged monitoring of the pupil of the eye, which system comprises a flexible or rigid transparent part (or base) in contact with the eye, like a contact lens, and an optoelectronic device fastened on said part and serving to collect optical information for use in measuring the pupil.

The diameter of the transparent part may lie in the range about 8 mm to about 18 mm, depending on whether it is applied against the cornea or against the sclera of the eye. If it is applied against the sclera, there may be a space between the cornea and the transparent part.

This part may incorporate the optical portion of the imager or light receivers in order to make the sensor thinner. Under such circumstances, the transparent base may incorporate microlenses, diaphragms, and/or micromirrors.

The system includes at least one source of light that is visible or in the near infrared, in particular having a wavelength situated in the range extending from about 700 nanometers (nm) to about 1000 nm, which light is used for illuminating the iris, and a light stimulation source that emits light that is white, red, green, or blue. The light receivers and the light sources face towards the pupil.

The system has electronic components that serve to provide proper operation of the system as a whole. The components may comprise passive components, and possibly active components (integrated circuits or microcontrollers) for managing the power supply of the sensor, for transmitting data, and for processing signals or data.

For comfort of the eyelid, the electrical or electronic components are covered (and protected) in a flexible or rigid covering that presents an ergonomic shape.

The optoelectronic device may be incorporated in the “contact lens”, or it may be fastened thereon in temporary manner, using a weak adhesive. When incorporated, the sensor is generally for single use, whereas when fastened in temporary manner, the contact lens may be for single use while the remaining portion of the sensor may be reusable.

The sensor as made up in this way communicates with an external module, via a plurality of conductors or else wirelessly.

With a wireless sensor, the energy needed by the electronic and optoelectronic components of the sensor may be delivered by induction: a power supply module placed in the proximity of the eyelid emits a modulated electromagnetic field that is picked up by an induction loop incorporated in the sensor, and the current that is induced in this loop is used to power the components of the sensor.

The sensor must also exchange data with the outside, generally in bidirectional manner.

The data may be transmitted via the induction loop or via a separate channel (radio or infrared).

In order to transmit information by radiation in the near infrared, use is made of the fact that the eyelid passes such radiation well: the signal for sending by the sensor is delivered by an infrared LED incorporated in the sensor and facing towards the eyelid. An infrared light receiver placed on an external module in the proximity of the eyelid receives the light signal emitted by the infrared LED after it has passed through the eyelid covering the sensor, and it recovers therefrom the information conveyed by the signal.

The operation is the same for infrared communication in the opposite direction: an infrared-emitting LED is placed on the external module facing towards the eyelid and a light receiver incorporated in the sensor receives the light signal after it has passed through the eyelid. The transmission techniques may be combined, e.g. by associating transmission by induction from the external module to the sensor, and infrared transmission from the sensor to the external module.

The external module serves to power the sensor, to control the sensor, and to receive (and possibly also to process) the information coming therefrom. The collected information comprises pupil image data or signals coming from light receivers placed in front of the pupil and making it possible to determine the dimensional characteristics thereof.

The means for processing this information serve to determine the parameters being monitored, in particular the size of the pupil, and to display those parameters on a user interface. The processor means may be incorporated in the external module or they may be remote in a separate system.

The sensor may be used to perform measurements at various frequencies, e.g. at a rate of several tens of measurements per second, or at a rate of one measurement per minute. When the measurement frequency is low, the sensor need not be powered between two successive measurements.

Light stimulation of the subject may be performed regularly and in discontinuous manner, e.g. once every minute, thereby enabling the photo motor reflex of the subject to be tested.

With a wireless sensor, the external communications module (emitter/receiver) may be placed in the proximity of the sensor, e.g. on the eyelid (with or without direct contact), thus making it possible to avoid integrating the lighting source and/or the light stimulation in the sensor under the eyelid.

Under such circumstances, it is also possible for the external communications module to form (or be incorporated in) a portable appliance that is moved up to the subject's eyelid in order to make a point measurement.

The electronic components of the sensor may contain a unique digital identifier that may be transmitted together with the measurement data or signals in order to identify the subject automatically, so as to be certain of associating the measurements that are taken with the subject in question.

The sensor may be arranged to measure other parameters such as performing oximetry of the back of the eye or measuring the subject's temperature.

It is also possible to use the sensor to detect or measure any movement of the eye under the eyelid. This may be of use in particular for predicting the awakening of an anesthetized subject. For this purpose, an external sensor such as a camera may be placed above the eyelid in order to identify the position of an infrared LED secured to the sensor that is placed under the eyelid. The movements of the infrared LED can be detected and measured by analyzing the images delivered by the external sensor, thereby providing information about eye movements.

The invention makes it possible to monitor the pupil of a patient with the eyes closed, and to do so safely. Once the sensor has been placed on the eye and the eyelid has been closed, no further human intervention is needed to monitor the activity of the eye; the subject may be stimulated by light stimulations or by pain stimulations (electrical, mechanical, or thermal).

In the description below, and unless indicated explicitly or implicitly to the contrary, elements or members that are structurally or functionally identical or similar are designated by identical references in the various figures.

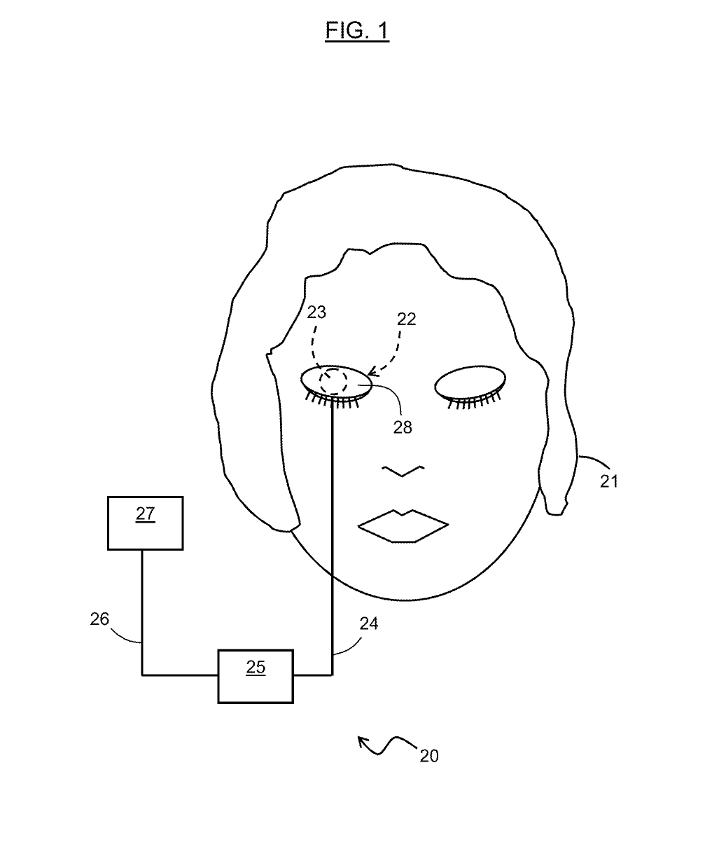

With reference to FIG. 1, a device 20 is used to monitor the size of the pupil of the right eye 22 of an unconscious subject 21 whose eyelids are closed.

For this purpose, the device 20 has a thin sensor 23 inserted under the upper eyelid 28 of the eye 22 and resting against the cornea and possibly also the sclera of the eye 22.

The device 20 includes a unit 27 for processing signals delivered by the sensor 23, e.g. a computer, which unit is arranged—in particular programmed—to determine the size of the pupil of the eye 22 as a function of the signals.

The device 20 also includes signal transport means connecting the sensor 23 to the signal processor unit 27 and enabling the subject's eyelids to be kept closed.

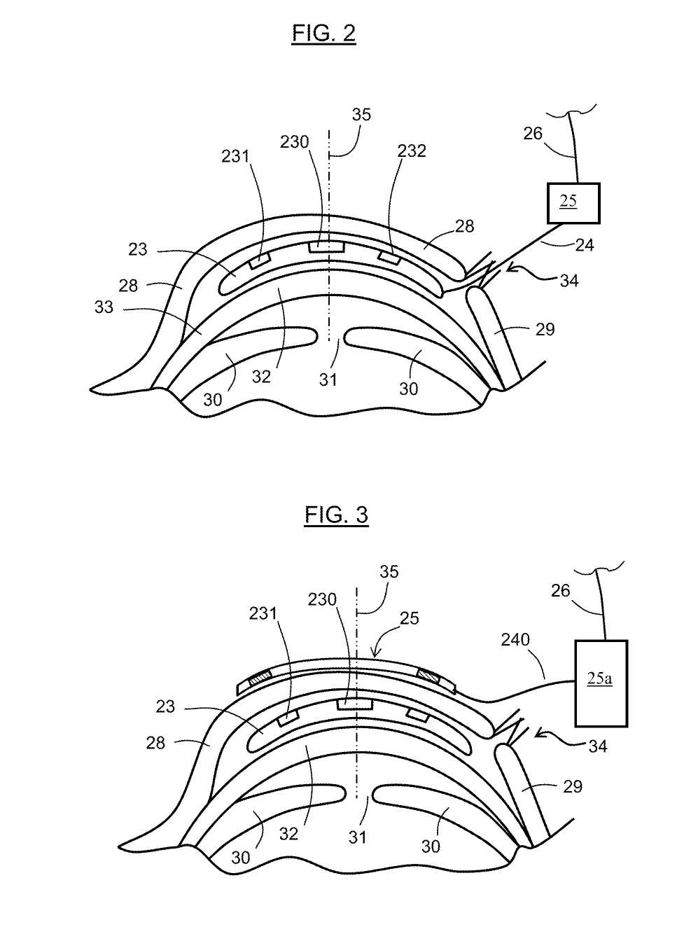

These transport means comprise an interface module 25 arranged in the proximity of the eye 22, a cable 24 connecting the sensor 23 to the interface 25 and extending, as shown in FIG. 2, through the gap 34 between the upper and lower eyelids 28 and 29 of the eye 22.

These transport means also include a cable 26 connecting the interface 25 to the processor unit 27.

With reference to FIGS. 2 and 3, the sensor 23 extends between the cornea 32 and the upper eyelid 28 that covers the sensor, in full or over a major fraction.

Depending on the size of the sensor body, the body may rest on the cornea alone, on the sclera 33, or else both on the cornea and on the sclera.

The body of the sensor is in the form of a spherical cap generally in the form of a body of revolution about an axis 35 passing through the pupil 31 as surrounded by the iris 30.

The sensor has light sensors 230 arranged centrally, i.e. substantially on the axis 35, stimulation LEDs 231 that emit in the visible range—e.g. white light—, and LEDs 232 for illuminating for measurement purposes, which LEDs 232 emit in the near infrared, for example.

The LEDs 231 and 232 are spaced apart from the axis 35, with it being possible for the illuminating LEDs 232 to be spaced further from said axis than the stimulation LEDs 231.

The receivers 230 are arranged in such a manner as to observe the central zone of the eye that includes the pupil 31, substantially on the axis 35.

The stimulation LEDs 231 are arranged so as to illuminate the back of the eye, and the illumination LEDs 232 are arranged so as to illuminate the iris.

In the embodiment of FIG. 3 in particular, in which the signals from the light receivers of the sensor 23 are transmitted through the eyelid 28 without equalizing potentials between the interface module and the sensor, i.e. “wirelessly”, the interface module includes a communications module 25 communicating with the sensor and a module 25a for formatting signals, which formatting module 25a is connected to the communications module by a cable 240.

The communications module 25 is arranged in the proximity of the eye, in particular on the upper eyelid 28 covering the sensor 23.

Under these circumstances in particular, the small distance between the module 25 and the sensor 23 may serve both for transmitting signals from the light receivers of the sensor to the module 25 (and thus to the module 25a and to the signal processor unit), and also for powering the components of the sensor by means of the module(s) 25, 25a and through the eyelid 28.

This near-field “communications” between the module 25 and the sensor 23 may take place using radio waves, by mutual induction between antennas (or antenna coils) incorporated respectively in the module 25 and the sensor 23, and/or by lightwaves (in particular infrared rays) passing through the eyelid 28.

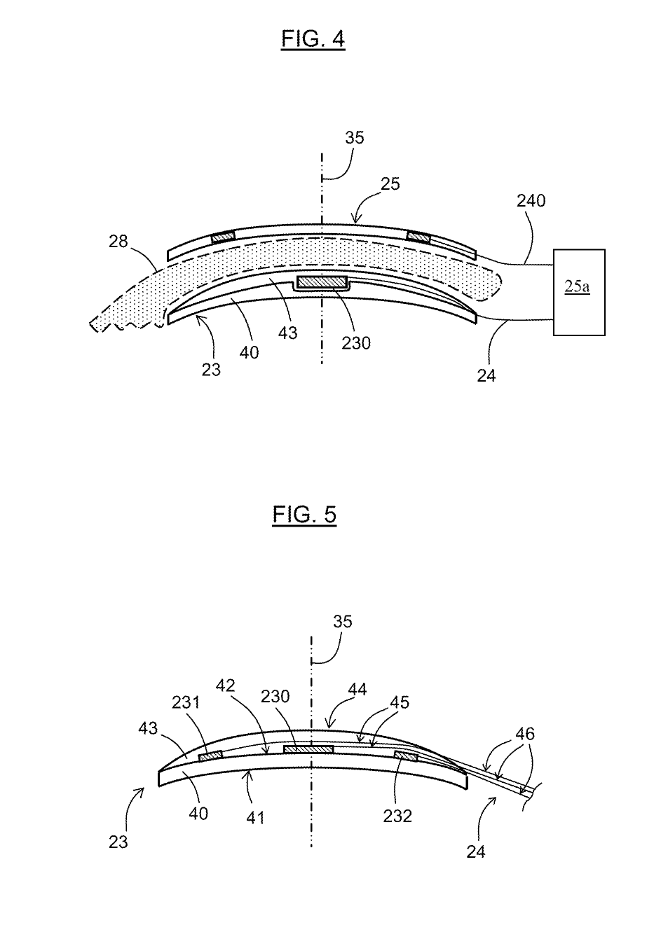

In the embodiment shown in FIG. 4, the interface and signal formatting module 25a is connected firstly to the sensor 23 by a cable 24 extending between the eyelids, and secondly to a communications module 25 resting on the eyelid 28, on its outside.

In the embodiments shown in FIGS. 5 to 8, in particular, the sensor 23 comprises a body 40, 43 extended by a ribbon cable 24.

The body of the sensor includes a proximal lens 40—in the form of a spherical cap—that is transparent to visible radiation and to infrared radiation, and that extends between a concave proximal face 41 and a convex distal face 42, each of these faces being a surface of revolution about the axis 35.

The receivers 230 and the LEDs 231, 232 are arranged on the distal face 42 of the lens 40 and they are covered by a second lens 43—or distal lens—constituting the covering, which may be secured to the lens 40, e.g. by adhesive.

The conductors 45 connected respectively to the LEDs and to the light receivers for the purpose of connecting them to the interface module are covered in part by the lens 43.

The portions 46 of these conductors that extend outside the body 40, 43 of the sensor, extending the body, are grouped together to form the cable 24.

The conductors 46 extend the conductors 45 that are protected by—or embedded in—the covering 43 that has a (distal) outer face 44 that is convex.

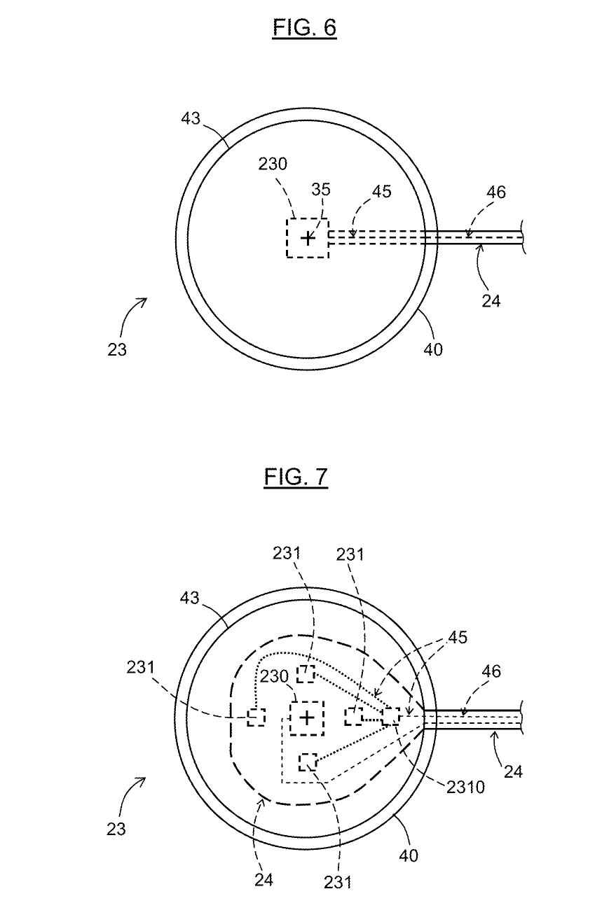

With reference to FIG. 6, the sensor 23 comprises a matrix imager 230 that may have several of light receivers—and possibly up to one or several millions of light receivers (or pixels)—, which imager is centered on the axis of symmetry 35 of the body 40, 43 of the sensor.

In the embodiment shown in FIG. 7, the sensor 23 induces a central matrix imager 230 and four stimulation LEDs 231 that are powered by the cable 24 extending the body 40, 43 via a distribution circuit 2310 connected for this purpose to the interface module (not shown).

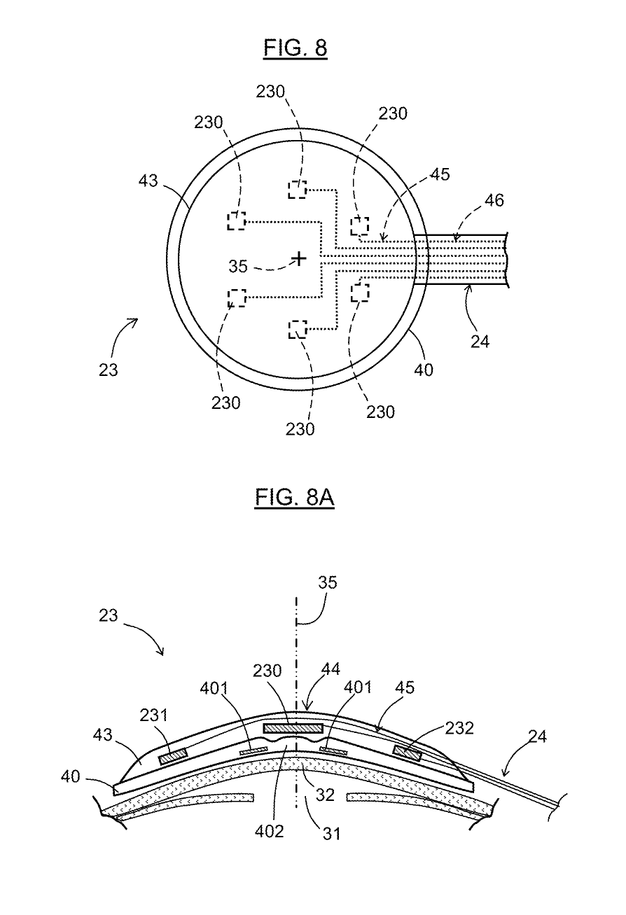

In the embodiment shown in FIG. 8, the sensor 23 has an array of “discrete” light receivers 230 distributed around the axis 35, each of which is associated with a miniature optical component such as a lens, whereby each light receiver observes only a portion of the iris and of the pupil.

With reference to FIG. 8A, a diaphragm 401 and a lens 402 are incorporated in the transparent base 40. The lens 402 is centered on the observation axis 35 of the imager 230 and serves to focus the imager in the plane of the pupil 31. The diaphragm 401, likewise centered on the axis 35, serves to optimize the optical properties of the system.

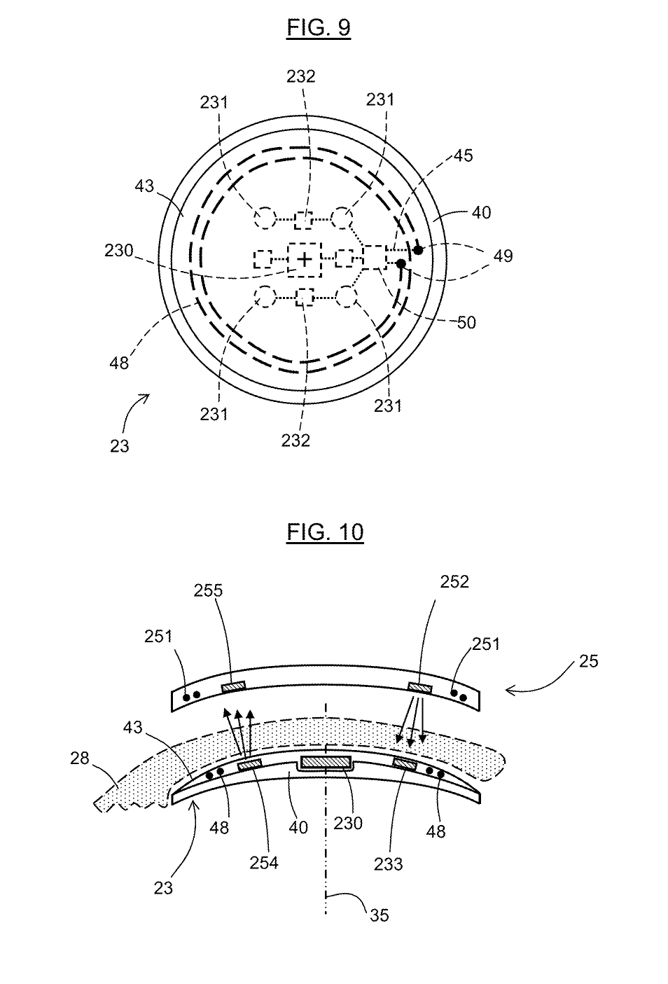

In the embodiment shown in FIG. 9, the sensor 23 has a spiral antenna coil 48 comprising two interleaved turns.

The terminals 49 of the antenna are connected to an integrated circuit 50 that serves to manage energy transmission for powering the components 230 to 232 of the sensor, and also for transmitting signals or data from the imager 230 to an external communications module (not shown).

For this purpose, the antenna 48, the integrated circuit 50, and the conductors 45 connecting the circuit 50 to the components 230 to 232, are integrated in the sensor and covered by the walls 40, 43 forming the body of the sensor.

With reference to FIG. 10, the transmission of data and the powering of the sensor through the eyelid 28 takes place over two different “channels”: the transmission of data from the imager 230 to the communications module 25 placed on the eyelid, takes place via light transducers 233, 254 (emitters and/or receivers) that are integrated in the sensor 23 and protected by the covering 43, through the covering and through the eyelid overlying the sensor, whereas the components 230 to 233 are powered by induction by means of a coil 48 incorporated in the sensor in similar manner to that shown in FIG. 9.

In this embodiment, the communications module 25 is provided for this purpose with an antenna 251 arranged to power the sensor by induction via the antenna 48, a receiver 255 that is sensitive to the radiation emitted by an emitter 254 of the sensor 23, and a light emitter 252 communicating with a receiver 233 of the sensor 23.

In this embodiment, the signal transmission device has emitters included in the body of the sensor that are connected to the light receivers and that are arranged to exchange light signals with the receivers of the external communications module.

Data transmission via the light transducers 233, 252, 254, and 255 may be obtained by radiation in a wavelength range for which the covering 43 and the eyelid 28 are not very opaque, and in particular in the infrared.

In a similar embodiment, the signal transmission device has an antenna included in the body of the sensor, the antenna being connected to the light receivers by conductors via an integrated circuit, and being arranged to exchange signals without contact (wirelessly) with the external communications module 25, 25a arranged in the proximity of the eye.

In another variant embodiment in which the sensor does not include any light source, as in the configurations shown in FIGS. 6 and 8, the light receivers are powered and the signals delivered by the light receivers are transmitted (wirelessly) by means of induction or by emitter/receivers forming parts of the sensor and of the external module 25, as shown in FIG. 10.

In addition, in this configuration in particular, the light stimulation of the eye and/or the illumination of the iris may be performed by light sources forming parts of the external module and producing light flux passing through the upper eyelid covering the eye.

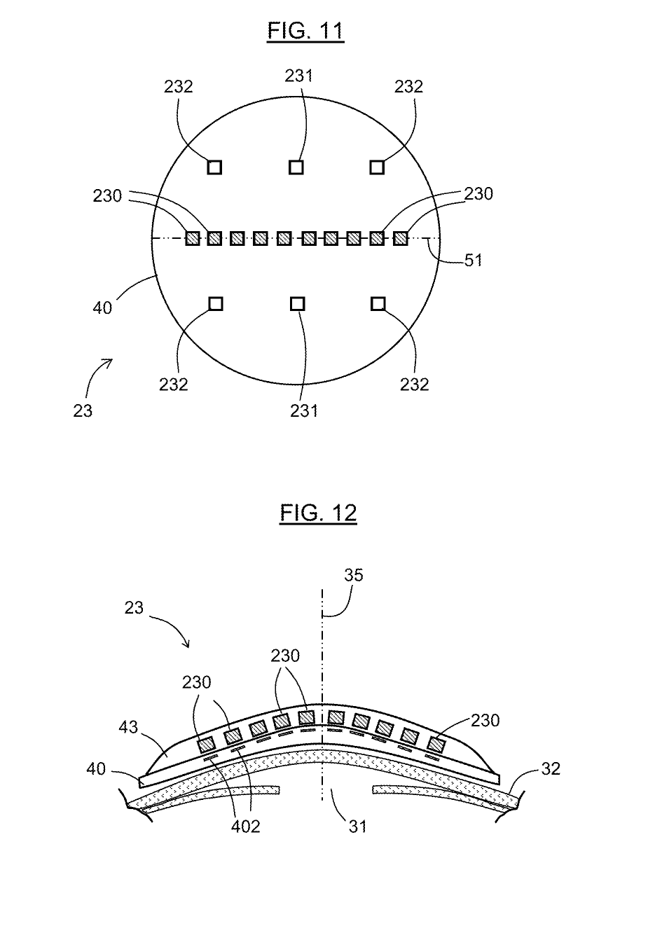

In the configuration shown in FIGS. 11 and 12, the sensor 23 has discrete (isolated) light receivers arranged in a line along a diameter 51 of a circle centered on the axis of symmetry of the body of the sensor.

This embodiment makes it possible to construct a “pseudo-image” (a low resolution image) on the basis of brightness information delivered by each light receiver, a microlens 402 being etched or otherwise arranged facing each light receiver (ideally etched in the contact lens).

This makes it possible to provide a sensor of smaller thickness and lesser power consumption, and to simplify the processing of the signals from the light receivers, thus enabling the processing to be performed more easily by an electronic circuit incorporated in the sensor. These advantages are particularly appreciable when using a wireless transmission sensor. Nevertheless, measurement accuracy may be less than that obtained with a matrix imager.

Each microlens serves to focus a small portion of the iris plane on the light receiver associated with the microlens.

This produces an in-line “image” of the diameter of the iris that corresponds to a brightness profile along a diameter of the iris, and that makes it possible to deduce the diameter of the pupil, as shown in FIG. 13.

With reference to FIG. 13, the position (NP) of the light receivers along the diametral “row” of light receivers is plotted along the abscissa axis, and the level (N) of the signals delivered respectively by the light receivers are plotted up the ordinate axis.

The light receivers that are placed in positions 1 to 4 and 8 to 10 observe the iris, and they deliver respective high level signals, whereas the light receivers that are placed in the positions 5 to 7 observe the pupil, and they deliver respective signals of lower level.

It is thus possible to estimate the diameter 60 of the pupil as being equal to the distance between the light receivers 4 and 8.

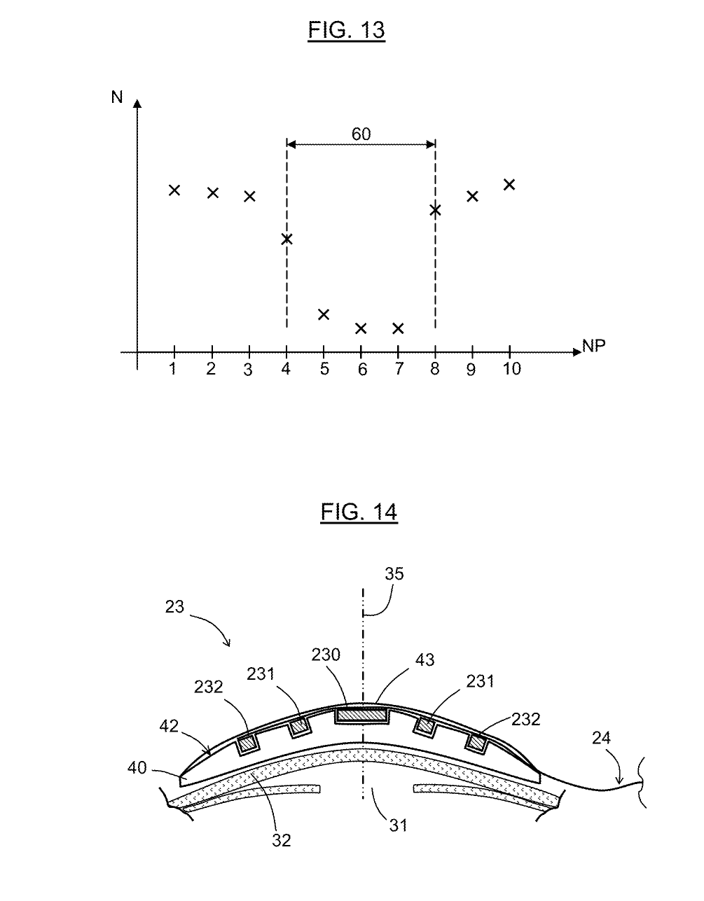

In the embodiment of FIGS. 14 to 16, a flexible printed circuit forms a ribbon cable 24 and carries an imager 230 and two pairs of LEDs 231, 232.

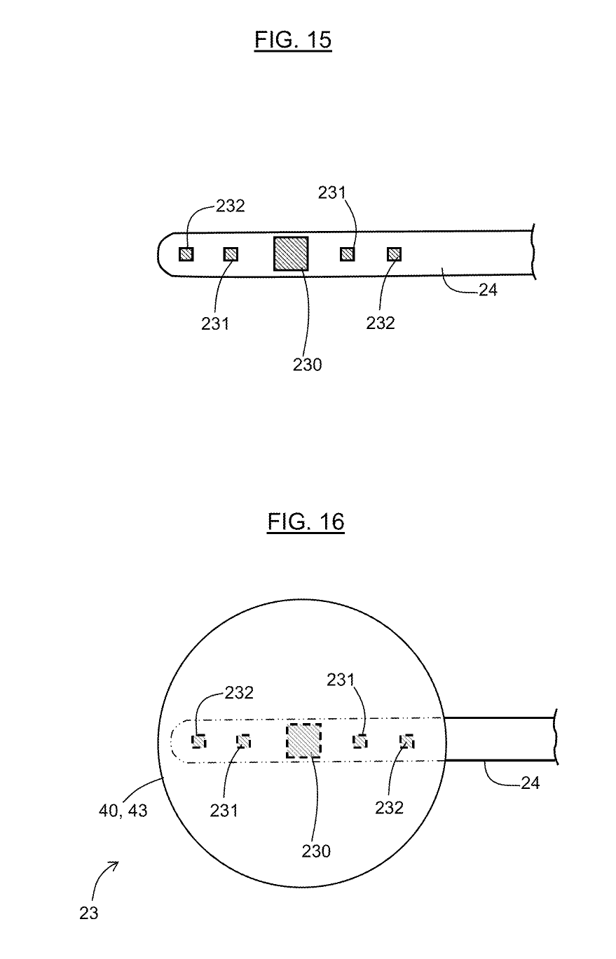

As shown in FIGS. 15 and 16, these components are arranged in line along the longitudinal axis of the circuit 24.

A sclera lens 40 has two cavities machined or molded in its distal face 42, with mutual spacings and dimensions adapted respectively to the spacing and dimensions of the components 230 to 232, such that these components can be inserted in the cavities.

A groove-shaped depression extends along a diameter of the lens 40 in the distal face of the lens and receives the portion of the flexible printed circuit that carries the components, with the remaining portion of the printed circuit extending radially away from the lens.

A leakproof covering film 43 covers the portion of the circuit that is housed in the depression together with the components 230 to 232.

1. A sensor (23) for monitoring the pupil (31) of an eye (22), the sensor being characterized in that it comprises:
a transparent base (40) presenting a concave first face (41) suitable for resting on the cornea (32) or the sclera (33), and a second face (42) opposite from the first face;
light receivers (230) arranged facing the second face of the transparent base in order to observe the pupil;
a signal transmission device (24, 45, 46, 48, 49, 50, 233) that is connected to the light receivers, that extends facing the transparent base or that extends said base, and that is arranged to take signals from the light receivers and transmit them through one or both of the eyelids (28, 29) covering the eye, and
a covering (43) covering the light receivers and at least a portion of the signal transmission device, the outer face (44) of the covering being adapted to be in contact with the closed eyelid(s).

2. A sensor according to claim 1, having a body including the transparent base (40) and the covering (43), wherein the body covers the light receivers (230) and conductors (45) connected to the light receivers, the body of the sensor being generally in the form of a spherical cap having two main outer faces: said concave first face (41) and a convex distal face (44) adapted to be in contact with the eyelid(s) covering the body of the sensor.

3. A sensor according to claim 1, wherein the base (40) comprises a soft or hard ophthalmic contact lens.

4. A sensor according to claim 1, wherein the base (40) and/or the covering (43) comprise(s) a leaktight coating of the light receivers.

5. A sensor according to claim 1, wherein the covering (43) is transparent to visible or infrared radiation.

6. A sensor according to claim 2, wherein the signal transmission device comprises a ribbon cable (24) extending the body of the sensor and containing conductors (46) extending conductors (45) contained in the body, the cable presenting thickness that is small enough to pass through the gap (34) between the closed eyelids, substantially without deforming or separating the eyelids.

7. A sensor according to claim 2, wherein the signal transmission device comprises an antenna (48) included in the body (40, 43) of the sensor and connected to the light receivers by conductors (45), where appropriate via an integrated circuit (50), the antenna being arranged to exchange signals with an external communications module (25, 25a) placed in the proximity of the eye.

8. A sensor according to claim 2, wherein the signal transmission device comprises emitters (233) included in the body (40, 43) of the sensor, which emitters are connected to the light receivers and are arranged to exchange light signals with receivers (252) of an external communications module (25, 25a) arranged in the proximity of the eye.

9. A sensor according to claim 1, including an optical system (401, 402) arranged between the concave face (41) of the transparent base and the light receivers (230) so as to focus the light receivers substantially on the plane of the pupil and the iris, and so as to limit the sensitivity of the light receivers to interfering radiation.

10. A sensor according to claim 1, including sources (231, 232) of visible light or of infrared radiation that are arranged facing the transparent base in order to illuminate the iris or to produce visible stimuli.

11. A sensor according to claim 10, wherein the sources (231, 232) of visible light or of infrared radiation are arranged in a peripheral portion of the sensor and are covered by the covering (43).

12. A sensor according to claim 1, including a matrix imager (230) having a large number of light receivers.

13. A sensor according to claim 2, wherein the thickness of the body (40, 43) of the sensor is less than or equal to four millimeters.

14. A sensor according to claim 13, including a matrix imager (230) having a large number of light receivers.

15. A device (20) for monitoring the pupil (31) of an eye (22) of a subject (21), the device including:
a sensor (23) adapted to be inserted between the eye and the eyelid(s),
a processor unit (27) for processing signals delivered by the sensor and arranged to determine the size of the pupil of the eye as a function of these signals, and
signal transport means (24, 25, 25a, 26) connecting the sensor to the signal processor unit and allowing the subject's eyelids (28, 29) to be kept closed.

16. A device according to claim 15, wherein the signal transport means comprise a wireless external communications module (25) arranged in the proximity of the sensor and including an antenna (251) and/or light transducers (252, 255).

17. A device according to claim 15, wherein the signal transport means comprise a wireless external communications module (25) arranged on the upper eyelid (28) covering the sensor (23), and including an antenna (251) and/or light transducers (252, 255).

18. A device according to claim 15, wherein the sensor comprises:
a transparent base (40) presenting a concave first face (41) suitable for resting on the cornea (32) or the sclera (33), and a second face (42) opposite from the first face;
light receivers (230) arranged facing the second face of the transparent base in order to observe the pupil;
a signal transmission device (24, 45, 46, 48, 49, 50, 233) that is connected to the light receivers, that extends facing the transparent base or that extends said base, and that is arranged to take signals from the light receivers and transmit them through one or both of the eyelids (28, 29) covering the eye, and
a covering (43) covering the light receivers and at least a portion of the signal transmission device, the outer face (44) of the covering being adapted to be in contact with the closed eyelid(s).

19. A method of monitoring the pupil of a subject, wherein a sensor for observing the pupil is arranged between the cornea and the closed eyelid(s), the sensor is powered through the eyelid(s), and pupil-observation signals delivered by the sensor are collected through the eyelid(s).

20. A method according to claim 19, wherein the sensor comprises:
a transparent base (40) presenting a concave first face (41) suitable for resting on the cornea (32) or the sclera (33), and a second face (42) opposite from the first face;
light receivers (230) arranged facing the second face of the transparent base in order to observe the pupil;
a signal transmission device (24, 45, 46, 48, 49, 50, 233) that is connected to the light receivers, that extends facing the transparent base or that extends said base, and that is arranged to take signals from the light receivers and transmit them through one or both of the eyelids (28, 29) covering the eye, and
a covering (43) covering the light receivers and at least a portion of the signal transmission device, the outer face (44) of the covering being adapted to be in contact with the closed eyelid(s).
